(19) United States
(12) Patent Application Publication
(10) Pub. No.: US 2026/0124127 A1
(43) Pub. Date: May 7, 2026

(54) BENEFICIAL SUNSCREEN

(71) Applicant: Stream2Sea, LLC, Bowling Green, FL (US)

(72) Inventor: Autumn Blum, Bowling Green, FL (US)

(73) Assignee: Stream2Sea, LLC, Bowling Green, FL (US)

(21) Appl. No.: 19/382,044

(22) Filed: Nov. 6, 2025

Related U.S. Application Data
(60) Provisional application No. 63/716,912, filed on Nov. 6, 2024.

(51) Int. Cl. A61K 8/29; A61K 8/04; A61K 8/19; A61K 8/20; A61K 8/23; A61K 8/27; A61K 8/34; A61K 8/365; A61K 8/9789; A61K 8/9794; A61Q 17/04

(52) U.S. Cl. CPC A61K 8/29; A61K 8/04; A61K 8/19; A61K 8/20; A61K 8/23; A61K 8/27; A61K 8/345; A61K 8/365; A61K 8/9789; A61K 8/9794; A61Q 17/04

(57) ABSTRACT

The present invention relates to skin care, sunscreen and cosmetic compositions that are environmentally friendly and have a beneficial impact on the health of coral when introduced into the environment.

### CROSS-REFERENCE TO RELATED APPLICATION

This application claims the benefit under 35 U.S.C. § 119 of U.S. Provisional Application Ser. No. 63/716,912, filed on Nov. 6, 2024, which is incorporated herein by reference.

### BACKGROUND OF THE INVENTION

### Field of the Invention

The present invention relates to cosmetic and skin compositions that are environmentally friendly and have a neutral to beneficial impact on the health of coral when introduced into the environment.

### Background of the Invention

The present invention encompasses a variety of cosmetic compositions, including but not limited to sunscreen, that are environmentally friendly and can have a neutral to beneficial impact on the health of coral when the compositions are introduced into the water around and near coral. The present application pertains to a unique blend of trace minerals that when added to mineral sunscreen compositions (or other cosmetic compositions) that are environmentally friendly and contain no petrochemical ingredients, can be considered beneficial to some coral.

While protection of the skin from ultraviolet rays is one of the major issues in skincare and body care, increasing efforts are being made to develop sunscreens that do not harm coral reefs. It has been established that many convention sunscreens negatively impact the health and growth of coral reefs. See, for example, National Academies of Sciences, Engineering, and Medicine. 2022. Review of Fate, Exposure, and Effects of Sunscreens in Aquatic Environments and Implications for Sunscreen Usage and Human Health. Washington, DC: The National Academies Press. https://doi.org/10.17226/26381 (last accessed on Oct. 10, 2024). Sunscreens accumulate in coral and can result in coral bleaching, damage to coral DNA, deformation of young coral, and even coral death. This invention relates to novel sunscreen compositions specifically designed to avoid harming and even enhance the health of coral reefs from, among other things, the harmful effects of traditional sunscreen chemicals. When combined with a sunscreen composition that comprises non-nano zinc oxide and/or titanium dioxide as the UV-blocking agents, which are known in the art to be reef-friendly, the invention's proprietary blend of trace minerals including magnesium, chloride, sodium, and potassium promote coral resilience and regeneration. Unlike conventional sunscreens that contain oxybenzone and octinoxate, chemicals known to contribute to coral bleaching, the formulations of the present invention have been tested in controlled environments and demonstrated significant increase in coral growth across various metrics compared to both control and formulas without the coral nutrients.

### BRIEF SUMMARY OF THE INVENTION

To facilitate understanding of the invention, the drawings and description illustrate preferred embodiments thereof, from which the invention, various embodiments of its compositions, structures, construction and method of operation, and many advantages, may be understood and appreciated. The drawings hereby are incorporated by reference.

The present application pertains to various compositions of blends of trace minerals that, when added to mineral sunscreen compositions (or other cosmetic compositions) that are environmentally friendly and contain no petrochemical ingredients, can be considered beneficial to coral. A low dose of one embodiment of the present invention has been shown to have a positive effect on corals in comparison to a control sunscreen treatment (6.3 μg/L) in Galaxea, which is a type of coral.

One embodiment of the present invention is a sunscreen composition comprising a UV-blocking agent and a blend of trace minerals that has a positive impact on the health of coral. In another embodiment of the present invention, the UV-blocking composition is selected from the group consisting of non-nano zinc oxide and/or titanium dioxide. In a third embodiment of the present invention, the blend of trace minerals comprises chloride, magnesium, sulfate, sodium, and/or potassium.

One embodiment of the present invention is a sunscreen composition comprising a sunscreen base and a trace mineral blend that has a positive effect on the health of at least one species of coral. For another embodiment, the sunscreen base comprises a UV-blocking composition is selected from the group consisting of non-nano zinc oxide and titanium dioxide. For one embodiment of the present invention, the trace mineral blend comprises chloride, magnesium, sulfate, sodium, and potassium. For one embodiment, the trace mineral blend comprises: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm. For one embodiment, the trace mineral blend comprises: (i) chloride in an amount from 340-550 mg/g of sunscreen composition, (ii) magnesium in an amount from 140-210 mg/g of sunscreen composition, (iii) sulfate in an amount from 15-60 mg/g of sunscreen composition, (iv) sodium in an amount from 2.5-9 mg/g of sunscreen composition, and (v) potassium in an amount less than 4 mg/g of sunscreen composition. For one embodiment of the present invention, the species of coral is selected from the group consisting of Porites lobata, Montipora capricornis, Galaxea fascicularis and Acropora muricata.

One embodiment of the present invention is a sunscreen composition comprising an oil phase comprising titanium dioxide in an amount from about 2.5% to about 25% titanium dioxide or zinc oxide in an amount from about 10% to about 25%, polyhydroxystearic acid, and a mixture comprising one or more esters. This embodiment also comprises an aqueous phase comprising water, a salt, a trace mineral blend in an amount from 0.1% to 5%, glycerol, an aloe compound, and green tea extract. For one embodiment of the present invention, the trace mineral blend comprises: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm.

One embodiment of the present invention is a sunscreen composition comprising an oil phase comprising titanium dioxide in an amount from 2.5% to about 25% or zinc oxide in an amount from 10% to about 25%, polyhydroxystearic acid, a mixture comprising one or more esters, and a trace mineral blend in an amount from 0.1% to 5%. For one embodiment of the present invention the trace mineral blend comprises: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm.

One embodiment of the present invention is a skin care composition comprising a skin care base; and a trace minder blend that has a positive impact on the health of at least one species of coral. For one embodiment, the skin care base is selected from the group consisting of a moisturizer, a toner, an essence, and a treatment. For one embodiment, the trace mineral blend comprises chloride, magnesium, sulfate, sodium, and potassium. For one embodiment the trace mineral blend comprises: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm.

One embodiment of the present invention is a method of making a skin care composition by combining a skin care base and a trace mineral blend, the trace mineral blend comprising: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm.

One embodiment of the present invention is a method of making a sunscreen composition by combining a sunscreen base and a trace mineral blend, the trace mineral blend comprising: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm.

One embodiment of the present invention is a sunscreen composition comprising a sunscreen base and a trace mineral blend comprising: (i) chloride in an amount from 200 to 600 ppm, (ii) magnesium in an amount from 100 to 300 ppm, (iii) sulfate in an amount from 2 to 100 ppm, (iv) sodium in an amount from 2 to 100 ppm, and (v) potassium in an amount from 0 to 100 ppm.

### BRIEF DESCRIPTION OF THE SEVERAL VIEWS OF THE DRAWINGS

To facilitate understanding of the invention, the drawings and description illustrate preferred embodiments thereof, from which the invention, various embodiments of its structures, construction and method of operation, and many advantages, may be understood and appreciated. The drawings are incorporated by reference.

FIG. 1 is a graph of sunscreen controls and embodiments of sunscreen compositions of the present invention and their impact on Acropora;

FIG. 2 is a graph of sunscreen controls and embodiments of sunscreen compositions of the present invention and their impact on Montipora;

FIG. 3 is a graph of sunscreen controls and embodiments of sunscreen compositions of the present invention and their impact on Galaxea; and

FIG. 4 is a graph of sunscreen controls and embodiments of sunscreen compositions of the present invention and their impact on Porites.

### DETAILED DESCRIPTION OF THE INVENTION

The following describes example embodiments in which the present invention may be practiced. This invention, however, may be embodied in many ways and the description provided herein should not be construed as limiting in any way. Among other things, the following invention may be embodied as compositions, systems, methods, or devices. The following detailed descriptions should not be taken in a limiting sense. The accompanying drawings are hereby incorporated by reference.

Before the example embodiments of the compositions and methods according to the present disclosure are disclosed and described below, it is to be understood that embodiments are not limited to those described within this disclosure. Numerous modifications and variations therein will be apparent to those skilled in the art and remain within the scope of the disclosure. It also is to be understood that the terminology used herein is for the purpose of describing specific embodiments only and is not intended to be limiting. Some embodiments of the disclosed technology will be described more fully hereinafter with reference to the accompanying drawings. This disclosed technology may, however, be embodied in many different forms and should not be construed as limited to the embodiments set forth therein.

In the following description, numerous specific details are set forth. However, it is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

If the specification states a component, element, part, or feature “may,” “can,” “could,” or “might” be included or have a characteristic, that particular component or feature is not required to be included or have the characteristic.

In this document, the terms “a” or “an” are used, as is common in patent documents, to include one or more than one. In this document, the term “or” is used to refer to a nonexclusive “or” such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

For purposes of the description hereinafter, it is to be understood that the specific compositions and methods illustrated in the attached drawings and tables in this specification are simply exemplary embodiments of the invention. Hence, specific compositions and other chemical characteristics related to the embodiments disclosed herein are not to be considered as limiting. Additionally, throughout this description reference is made to a sunscreen base 150 and a trace mineral blend 110. This separation of these components does not indicate that they are necessarily formulated separately and then mixed. As will be obvious to one skilled in the art, depending upon the composition of the sunscreen base 150 the trace mineral blend 110 can be added at an appropriate time during manufacture or formulation. For some sunscreen compositions 100 (and some skin care compositions 200) the trace mineral blend 110 will need to be added with certain ingredients and at certain times during manufacture to ensure that the trace mineral blend 110 mixes with the base 150 or 250. One skilled in the art will understand how and when to incorporate a solid/powder form or an aqueous form of a trace mineral blend 110 with the ingredients of a sunscreen base 150 or a skin care base 250 in order to formulate an effective sunscreen composition 100 or a skin care composition 200 of the present invention.

The materials described hereinafter as making up the various elements of the embodiments of the present disclosure are intended to be illustrative and not restrictive. Many suitable materials that would perform the same or a similar function as the materials described herein are intended to be embraced within the scope of the example embodiments. Such other materials not described herein can include, but are not limited to, materials that are developed after the time of the development of the invention, for example.

While the disclosure has been described in detail and referring to specific embodiments thereof, it will be apparent to one skilled in the art that various changes and modifications can be made without departing from the spirit and scope of the embodiments. Thus, it is intended that the present disclosure covers the modifications and variations of this disclosure provided they come within the scope of the appended claims and their equivalents.

Compositions and Formulations. The present invention encompasses a variety of cosmetic, skin, and sunscreen compositions that are environmentally neutral or friendly and can have a neutral or beneficial impact on the health of coral when the compositions are introduced into the water around and near coral. The present invention pertains to a trace mineral blend 110 that, when added to sunscreen bases 150 (or other skin care or cosmetic base compositions) that are environmentally friendly (or of neutral environmental impact and contain no petrochemical ingredients) can be considered neutral to most coral and beneficial to specific types of coral. While the following examples are sunscreen compositions 100, various embodiments of the present invention comprise the addition of a trace mineral blend 110 to a wide variety of cosmetics and skin care base compositions 250 to create products that are neutral to beneficial to coral when introduced into the environment around coral. The non-limiting example embodiments of the present invention relate to sunscreen compositions 100. The term “sunscreen base 150” is used herein to encompass suntan lotions, sunscreens, skin protectants, and sunblockers, which are intended for use on the face and/or body to provide protection against the sun's rays or other UV sources. Many of the sunscreen bases 150 of the present invention comprise non-nano zinc oxide, titanium dioxide, and/or other reef-friendly UV-blocking agents in combination with a trace mineral blend 110 to create a novel sunscreen composition 100 of the present invention. Many of these sunscreen bases 150 are formulations known to those in the art. These sunscreen compositions 100 include various embodiments of a trace mineral blend 110, which can include magnesium, chloride, sodium, sulfate, and potassium, which in combination have a neutral impact on most types of coral and a positive impact on the overall health of specific types of coral, including but not limited to improvements in resilience, regeneration, symbiotic balance, and skeletal growth.

For the purposes of this invention, the novel sunscreen compositions 100 (and the methods of making them 300) comprise a sunscreen base 150 and a trace mineral blend 110. One embodiment of the present invention is a method of incorporating a trace mineral blend 110 into a sunscreen base 150 or skin care base 250, which base 150, 250 independently has a neutral, or nonharmful impact, on coral to create a sunscreen composition 100 or skin care composition 200 that has a positive impact on at least some coral species. For various embodiments of the present invention, a skin care base 250 can be a skin moisturizer (balm, cream, lotion, or oil), a skin care toner, a skin essence, and/or a skin care serum or treatment. Again, for various embodiment of the novel sunscreen composition 100 of this invention, the sunscreen base 150 only needs to be formulated to not have a harmful impact on coral. Such formulations are known to one in the art. The example formulations provided herein (and methods of making them 300) are intended to be non-limited examples of various sunscreen compositions 100 according to the invention. Similarly, for non-sunscreen skin care compositions 200 according to the present invention, the skin care base 250 is required to have a neutral, or non-harmful, impact on the health of coral. Many such skin care bases are known to one skilled in the art. The present invention comprises the incorporation of a trace mineral blend 110 into a sunscreen base 150 or a skin care base 250 to produce a sunscreen composition 100 or a skin care composition 200 that has a positive impact on the health of certain types of coral when the sunscreen composition 100 or skin care composition 200 in introduced into the water surrounding those species of coral.

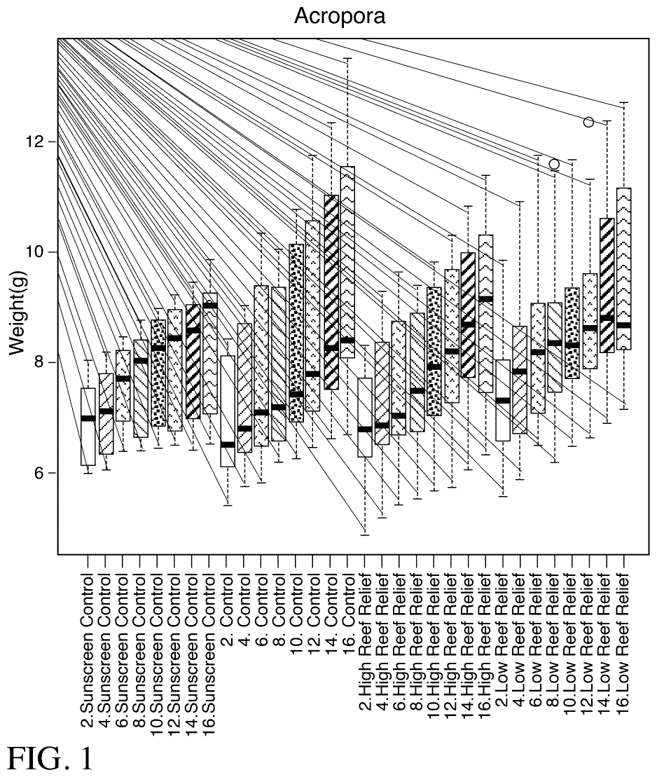

In one embodiment of the present invention, the sunscreen composition 100 is an emulsion. The emulsion of this embodiment comprises an oil phase and an aqueous phase. The oil phase comprises from about 2.5% to about 25% titanium dioxide and/or about 10% to about 25% zinc oxide, polyhydroxystearic acid, and a mixture comprising one or more esters. The aqueous phase comprises water, a salt, a trace mineral blend 110, glycerol, an aloe compound, and green tea extract. For this embodiment, about 0.1% to 5% of a trace mineral blend 110 is added. Advantageously, the novel sunscreen compositions 100 described herein have a surprising and unexpected ability to promote coral growth and increase thermal resilience, as shown by the graphs in FIGS. 1 through 4 (discussed more fully herein.)

In another embodiment of the present invention, the sunscreen composition 100 (and methods of making them 300) comprises an anhydrous mixture. The mixture of this embodiment comprises an oil phase comprising from about 2.5% to about 25% titanium dioxide and/or 10% to about 25% zinc oxide, polyhydroxystearic acid, a mixture comprising one or more esters, and 0.1% to 5% of a trace mineral blend 110.

The present invention incorporates 0.1% to 5% of a trace mineral blend 110 with a sunscreen base 150 or a skin care base 250. The amount of trace mineral blend 110 can exceed 5% and the percentage is only limited by the ability to incorporate the trace mineral blend 110 into a sunscreen base 150 or skin care base 250 without compromising the integrity and performance of the sunscreen base 150 or the skin care base 250.

Another embodiment of the present invention comprises a water resistant sunscreen composition 100. One embodiment of the present invention is a sunscreen composition 100 that includes an embodiment of the trace mineral blend 110 in an otherwise reef-safe, water resistant sunscreen base 150. The FDA establishes standards for a sunscreen bases 150 to be considered “water resistant”. Those standards include passing a water-resistant test and providing sun protection on wet skin for 40- to 80-minutes. There are many ingredients known in the art that make a sunscreen composition water resistant, including but not limited to, certain plant oils, alcohol, and beeswax.

Another embodiment of the present invention is a lotion or cream sunscreen composition 100 comprising a reef-safe sunscreen base 150 with an effective amount of a trace minder blend 110. Lotion or cream sunscreen compositions 100 are the most traditional and reliable form of sunscreen application, allowing users to evenly distribute the sunscreen over exposed skin. A highly water resistant formulation can ensure better adherence to the skin and provide a longer-lasting barrier that reduces the need for frequent reapplication. This method ensures that the product remains in contact with the user's skin for an extended period, minimizing the amount washed off into the marine environment.

Another embodiment of the present invention is a method 300 of incorporating an effective amount of a trace mineral blend 110 into a balm or stick sunscreen base 150 application. An anhydrous balm or stick application would allow users to apply the sunscreen composition 100 to specific areas of the skin with precision. This method 300 is ideal for users participating in water activities (e.g., snorkeling, diving) where extended water exposure might otherwise reduce effectiveness. The compact form is convenient, reduces waste, and prevents excess product from washing off into the water.

The present invention also encompasses an environmentally-friendly spray sunscreen compositions 100 which incorporates an effective amount of a trace mineral blend 110 into spray sunscreen base 150. While sprays are popular for ease of use, the typical aerosol cans are not environmentally friendly due to propellants and the risk of overspray entering the environment. However, a reef-protective sunscreen composition 100 can be formulated in a pump-spray or non-aerosol mist that ensures an even application while limiting the environmental impact. Newer technologies, such as “BOV” or Bag On Valve, which use compressed gas around a pouch, separating the sunscreen from the propellant can be configured for use with sunscreen compositions 100 of the present invention. This enables quick, widespread coverage without excessive product loss into the surrounding environment.

One embodiment of the sunscreen composition 100 comprises the sunscreen base 150 and the trace mineral blend 110, wherein the sunscreen composition 100 is configured as a spray formulation comprising a biodegradable carrier selected from the group consisting of medium-chain triglycerides, sugar-derived esters, and plant-based emollients, and wherein the composition is delivered through a non-aerosol or bag-on-valve delivery system.

One embodiment of the sunscreen composition 100 comprises the sunscreen base 150 and the trace mineral blend 110, wherein the sunscreen composition 100 is water-resistant for at least 80 minutes in accordance with any regulations, including but not limited to FDA 21 CFR 352.76, and maintains a neutral to beneficial impact on the health of coral after exposure to simulated seawater conditions.

Unique Trace Mineral Blends 110. Various embodiments of the trace mineral blend 110 of the present invention comprise a blend of minerals that have been shown to have a positive impact on the health of certain species of coral. Again, an effective amount of a trace mineral blend 110 is about 0.1% to 5% of the final composition, but the amount can exceed 5% if the base composition's effectiveness and integrity are not compromised by the inclusion of the trace mineral blend 110. The exact composition of the trace mineral blend 110 can vary and different compositions of the blend 110 will have a neutral to positive impact on the health of most species of coral. The blend 110 can be customized to specific types of coral that are located in specific geographic areas. Alternatively, the blend 100 can be customized to minimize interference with the efficacy of the sunscreen base 150 or skin care base 250 into which it is blended. One embodiment of a trace mineral blend 110 is a high potency, standardized mineral and trace mineral complex comprising chloride, magnesium, sulfate, sodium and potassium.

The various embodiments of a trace mineral blend 110 of the present invention can be a solid or powder form or it can be a water soluble, aqueous form of the minerals. One embodiment is free from (or has very low amounts of) heavy metals and microbial contamination. Tables 1 and 2 shows some non-limiting examples of the effective amounts of minerals (effective proportions) in some embodiments of a trace mineral blend 110 of the present invention, which can be incorporated into a sunscreen base 150 or a skin care base 250.

TABLE 1
Trace mineral blends 110

| Minerals | mg/g or ppm |
|---|---|
| Chloride | 200-600 |
| Magnesium | 100-300 |
| Sulfate | 2-100 |
| Sodium | 2-100 |
| Potassium | 0-100 |

TABLE 2
Trace mineral blends 110

| Minerals | Specification (ppm) | |
|---|---|---|
| Chloride | 340-550 | mg/g |
| Magnesium | 140-210 | mg/g |
| Sulfate | 15-60 | mg/g |
| Sodium | 2.5-9 | mg/g |

| | |
|---|---|
| Potassium | Less than 4 mg/g |

When using or preparing trace mineral blends 110 of the present invention, some embodiments are formulated to limit heavy metals to the amounts shown in Table 3. Also, to facilitate the combining of a trace mineral blend 110 into a sunscreen base 150 or a skin care base 250 while minimizing the impact on the base 150 or 250, the physical properties of the trace mineral blend 110 can be configured according to the limitations shown in Table 4 for certain embodiments. Additionally, some embodiments of a trace mineral blend 110 can be formulated to have the desirable limitations detailed in Table 5.

TABLE 3

| Key Heavy Metals | Specification | |
|---|---|---|
| Arsenic (Inorganic) | <10 | ppm |
| Lead | <0.5 | ppm |
| Mercury | <0.1 | ppm |
| Cadmium | <0.5 | ppm |

TABLE 4

| Physical Properties | Specification |
|---|---|
| Color | White |
| Moisture Content | <15% |
| pH | 8.5-9.8 |

TABLE 5

| Microbiology | Method | Specification |
|---|---|---|
| Standard Plate Count | AOAC 966.23 | Less than 1000 CFU/g |
| | or USP | |
| Escherichia Coli | AOAC 991.14 | Less than 100 cfu/g |
| | or FDA BAM | or <10 MPN/g |
| Coliform | AOAC 991.14 | Less than 10 CFU/g |
| | or FDA BAM | or <10 MPN/g |
| | or USP | or Absent per |
| | <2022> | |
| Staphylococcus | AOAC | Less than 100 cfu/g or |
| Aureus | 2003.07 or | Absent per 10 g |
| | USP <2022> | |
| Salmonella | Modified | Negative per 25 g or |
| | AOAC 998.09 | Absent per 10 g |
| | or FDA BAM | |
| | or USP | |
| | <2022> | |
| Yeast | FDA BAM or | 5100 cfu/g |
| | USP <2021> | |
| Mold | FDA BAM or | ≤100 cfu/g |
| | USP <2021> | |

According to embodiments of the present invention, a trace mineral blend 110 should be chosen so as to not disrupt or lessen the effectiveness of the sunscreen base 150 or skin care base 250 and so that the trace mineral blend 110 is at an amount or percentage of the product that has a beneficial impact on the health of aquatic life, such as certain species of coral, when it is introduced into the aqueous environment surrounding the aquatic lifeform. Some non-limiting examples of minerals that can be incorporated into the trace mineral blend 110 to achieve the desired effect on coral health include chloride, magnesium, sulfate, sodium, potassium, boron, and lithium. In other embodiments, antioxidants can be included for further reef care. Other minerals and antioxidants that satisfy the stated goals, are not harmful to humans, and do not decrease the effectiveness of the underlying base compositions are included within the scope of this invention.

Non-limiting Examples of Sunscreen Compositions 100. As explained previously, the present invention comprises the combination of various embodiments of a trace mineral blend 110 into any reef-safe sunscreen base 150 or skin care base 250. It will be obvious to one skilled in the art that any of a wide variety of sunscreen bases 250 can be used for this invention with the primary requirement that the sunscreen base 150 itself be either reef-friendly or neutral in its impact on coral health. The addition of a trace mineral blend 110 to a sunscreen base 150 that meets these criteria creates a sunscreen composition 100 with coral benefits. The following are some non-limiting examples of sunscreen bases 150 that can be combined with a trace element blend 110.

Some embodiments of a sunscreen base 150 are configured with an oil phase comprising from about 15 to about 25% zinc oxide, polyhydroxystearic acid, and a mixture comprising one or more esters. The ingredients in the oil phase may generally be mixed in any convenient order which order may vary depending upon the specific ingredients, amounts, and mixing method.

The zinc oxide active ingredient in some sunscreen bases 150 can be present in amounts which vary depending upon the level of SPF protection desired, other ingredients, and other desired characteristics of the sunscreen. Typically, a zinc oxide active ingredient can be present in at least about 15, or at least about 17, or at least about 18 up to about 25, or up to about 23, or up to about 21 weight percent based on the total weight of the sunscreen composition. In some embodiments the zinc oxide active ingredient is present at from about 18.5 to about 19.5 weight percent.

The polyhydroxystearic acid (or another plant based dispersant) is generally employed in an emulsifying amount. Such amounts may vary depending upon the other ingredients but often may be from about 0.5, or from about 1, or from about 2, up to about 5, or up to about 7 percent weight based on the total weight of the sunscreen composition 100.

The mixture comprising one or more esters is not particularly limited. In some embodiments the mixture comprising one or more esters comprises coco-caprylate/caprate, isoamyl laurate, or any mixture thereof. Additionally, or alternatively, in some embodiments the mixture comprising one or more esters may comprise a jojoba ester, a polyglyeryl ester such as polyglyceryl-3-oleate, or any mixture thereof. As with the other ingredients the amount of esters in the formulation varies depending upon the desired characteristics and other ingredients. In some cases, the amounts may be from about 10, or from about 14, or from about 19 up to about 25 or up to about 20 weight percent based on the total weight of the sunscreen composition 100.

The oil phase comprises optional additional components such as, for example, from about 0.1 to about 1% or up to about 3% weight percent based on total weight of a hydrogenated triterpene like squalane oil. The oil phase can also optionally comprise mica, silica, or a mixture thereof in amounts of from about 1 to about 3 weight percent based on the total weight of the sunscreen composition 100.

Some sunscreen bases 150 of the present invention have an aqueous phase of that generally comprises water, such as distilled water, a salt, glycerol, and optionally an aloe compound, and/or Curcuma longa (turmeric) root. The amount of water may vary but in some embodiments is more than 30, or more than 35 or more than 40 up to about 60, or up to about 50, or up to about 45 weight percent based on total weight of the sunscreen composition 100.

The salt is not particularly limited and may comprise, for example, NaCl in an amount of at least about 0.5, or at least about 1 up to about 5, or up to about 2, or up to about 1.5 weight percent based on total weight of the sunscreen composition 100.

The aloe compound, if present, can be selected from any available compound and in some embodiments may comprise Aloe Barbadensis Leaf Juice in a reconstituted amount of from about 0.05 up to about 0.2, or from about 0.2 up to about 30 weight percent based on total weight of the sunscreen composition 100.

The aqueous phase can further comprise other ingredients such as a glycol like propylene glycol, a sugar alcohol, a preservative and/or chelating agent like ethyl lauroyl arginate hydrochloride, tetrasodium glutamate diacetate, potassium sorbate, sodium stearoyl glutamate and any mixtures thereof

The sunscreen bases 150 of the present invention can, optionally, comprise other ingredients such as mixed tocopherols, film formers such as diisostearoyl polyglyceryl-3 dimer dilinoleate, Caprylic/Capric Triglyceride, and any mixture thereof.

One embodiment of a sunscreen composition 100 according to the present invention is illustrated in Table 6. The method of making 300 this composition 100 entails mixing the identified ingredients.

TABLE 6

| | | KG in |
|---|---|---|
| Component | % | Batch |
| Sunscreen Base 150 | 0.988 | 10.374 |
| Trace Mineral Blend 110 | 0.012 | 0.126 |
| Sunscreen Composition 100 | 1.000 | 10.500 |

Another embodiment of a sunscreen composition 100 illustrated in Table 7 along with some manufacturing instructions for a method of making 300 this composition 100.

TABLE 7

| | | KG in |
|---|---|---|
| Raw Material | % | Batch |
| Zinc sunscreen base | 0.99 | 149.16 |
| 150 | | |
| Trace Mineral blend | 0.01 | 0.84 |
| in powder form 110 | | |
| Sunscreen | 1.00 | 150.00 |
| composition 100 | | |

Warm up to fluid. Record temp
Add Powder and homogenize
Pour into containers and chill rapidly

| | |
|---|---|
| Trace mineral | |
| solution = 60% water | |
| Trace minerals | 0.10-2.00% |
| powder | |

Another embodiment of a sunscreen base 150 is detailed in Table 8. For this embodiment the sunscreen base 150 has an SPF of 30 and the batch size is 1200 kg. A trace mineral blend 110 (according to Tables 1 or 2) can be combined with the base 150 detailed in Table 8 to make a sunscreen composition 100. The method of making 300 this composition 100 entails mixing the identified ingredients as identified in Tables 8 and 10.

TABLE 8

| | | BATCH SIZE: | |
|---|---|---|---|
| | | 1200 | kg |
| Phase | Raw Material | % | KG in Batch |
| A | Deionized Water | 51.65 | 619.80 |
| B | Aloe ORGANIC | 10.00 | 120.00 |
| B | Botanical Extracts including green tea | 1.00 | 12.00 |
| C | Plant Based Emulsifiers | 6.00 | 72.00 |
| D | Plant Based Emollients | 15.00 | 180.00 |
| E | Polyhydroxystearic Acid | 2.00 | 24.00 |
| E | Titanium Dioxide | 8.00 | 96.00 |
| F1 | Glycerin | 3.00 | 36.00 |
| F1 | Xanthan Gum | 0.30 | 3.60 |
| F2 | Preservative | 1.00 | 12.00 |
| F2 | Film Former | 2.00 | 24.00 |
| F3 | Lactic Acid | 0.05 | 0.60 |
| | pH target: 5.0-5.4 | 100.00 | 1200.00 |

Another embodiment of a sunscreen base 150 is detailed in Table 9. For this embodiment the zinc balm sunscreen base 150 has an SPF of 40 and the batch size is 214 kg. A trace mineral blend 110 (according to Tables 1 or 2) can be combined with the base 150 detailed in Table 9 to make a sunscreen composition 100. The method of making 300 this composition 100 entails mixing the identified ingredients as detailed in Table 9.

TABLE 9

| | OIL CONCENTRATE | |
|---|---|---|
| | Batch Size | |
| | 214 | kgs |
| Raw Material | % | KG in Batch |
| Squalane | 0.40 | 0.8560 |
| Helianthus Annuus (Sunflower) Seed Oil | 28.50 | 60.9900 |
| Prunus Amygdalus Dulcis (Sweet Almond) Oil | 27.00 | 57.7800 |
| Jojoba Esters | 2.00 | 4.2800 |
| Beeswax | 15.00 | 32.1000 |
| Sorbitan Olivate | 2.00 | 4.2800 |

Cool to less than 140-degrees for packaging.

| | | |
|---|---|---|
| Zinc Oxide, polyhydroxystearic acid | 22.10 | 47.2940 |
| Mica (and) Silica | 2.00 | 4.2800 |
| Tocopherols | 1 | 2.1400 |
| | 100.000 | 214.00 |

TABLE 10

| Raw Material | % |
|---|---|
| Stream2Sea SPF 30 Base | 0.990 |
| Trace Minerals Liquid | 0.010 |
| Total | 1.000 |

Another embodiment of a sunscreen base 150 is detailed in Table 11. For this embodiment the zinc balm sunscreen base 150 has an SPF of 40 and the batch size is 214 kg. A trace mineral blend 110 (according to Tables 1 or 2) can be combined with the base 150 as detailed in Table 12 to make a sunscreen composition 100. The method of making 300 this composition 100 entails mixing the identified ingredients as detailed in Tables 11 and 12.

TABLE 11

BATCH SIZE:

| | 214 | kgs |
|---|---|---|
| Raw Material | % | KG in Batch |
| Squalane | 0.40 | 0.8560 |
| Sunflower Oil, HO | 27.80 | 59.4920 |
| Sweet Almond Oil | 28.60 | 61.2040 |
| Floraesters 70 | 3.00 | 6.4200 |
| Beeswax-White | 13.60 | 29.1040 |
| Olivem 900 | 1.50 | 3.2100 |

To facilitate packaging cool to less than 140-degrees F.

| | | |
|---|---|---|
| Zinc Sheer Natural | 22.10 | 47.2940 |
| NS Boost | 2.00 | 4.2800 |
| Tocopherols | 1 | 2.1400 |
| | 100.000 | 214.00 |

TABLE 12

| | BATCH SIZE: |
|---|---|
| | 150 |
| | grams |
| Raw Material | % |
| Zinc Balm Base 150 | 0.99 |
| Trace Minerals Powder | 0.01 |
| 110 | |
| Sunscreen Composition | 1.00 |
| 100 | |

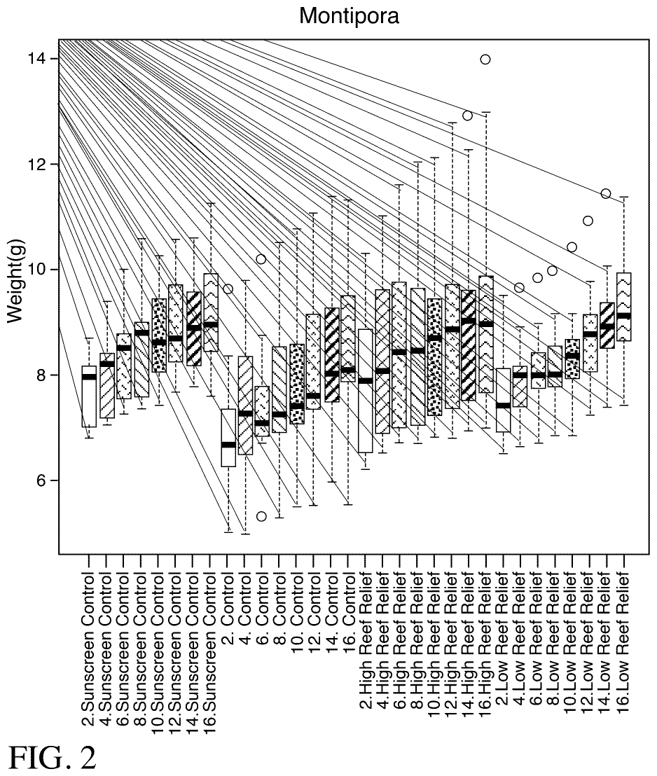

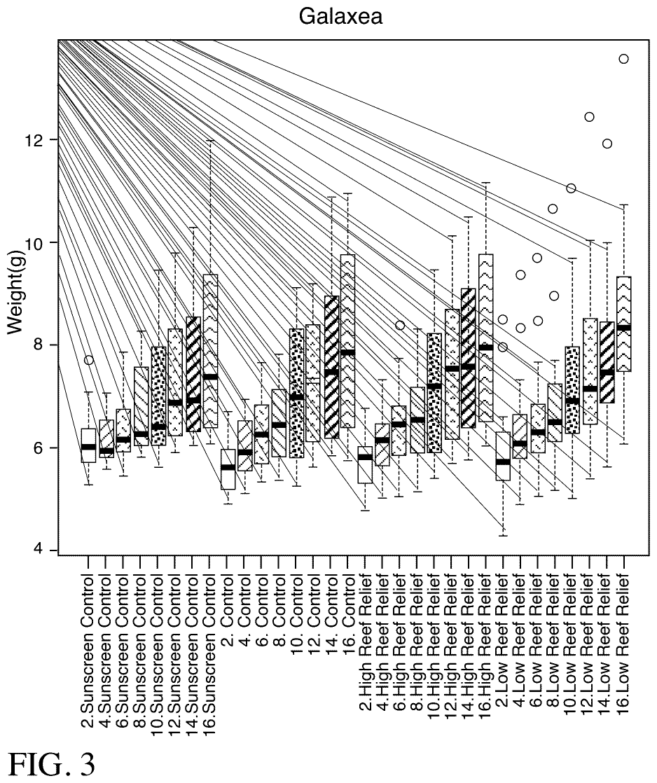

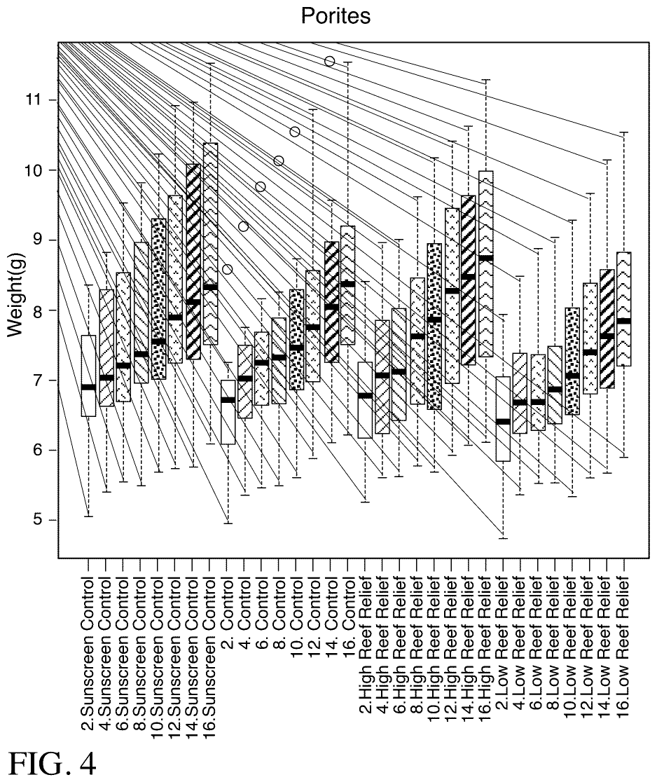

Research and Results. FIGS. 1 through 4 and Tables 13-16 illustrate the results of research on the impact of some embodiments of the present invention sunscreen compositions 100 on various forms of coral, specifically Acropora (FIG. 1 and Table 13,) Montipora (FIG. 2 and Table 14,) Galaxea (FIG. 3 and Table 15,) and Porites (FIG. 4 and Table 16.) For the data presented in FIGS. 1 through 4 and Tables 13-16, the weights were analysed with a GLS (Generalised Least Squares framework) because of heterogeneity of variance within the data. The t-values indicate the direction of the results (e.g. a positive number indicates a positive growth). The p-values indicate significance which was pre-determined before analysis. In this case, the desired significance for the standard was p<0.05. The intercept is the treatment the others are compared against, so the treatment that is not labelled is the comparison. As shown in FIG. 3 and Table 15, a low dose of one embodiment of the sunscreen composition 100 of the present invention has a positive effect on corals in comparison to the control sunscreen treatment (6.3 μg/L) in Galaxea. A “positive effect” or “positive impact” can be represented by an increase in any of the following attributes: horizontal growth, vertical, and/or weight, or by having a less negative impact than the sunscreen base 150 without the inclusion of the trace mineral blend 110. More broadly, a positive effect or impact on coral health means that the trace minerals in combination have a neutral impact on most types of coral and a positive impact on the overall health of specific types of coral, including but not limited to improvements in resilience, regeneration, symbiotic balance, and skeletal growth. As illustrated in FIG. 1 and Table 13, the control sunscreen significantly inhibits Acropora growth in comparison to the control treatment which had nothing added.

TABLE 13
Acropora Weight Results

| | Value | Std. Error | t-Value | p-Value | Notes |
|---|---|---|---|---|---|
| (Intercept) | 2.6970000 | 0.4560496 | 5.913830 | 0.0000 | There are no significant differences |
| Treatment High | −0.5850000 | 0.5068307 | −1.154232 | 0.2554 | between the control and the reef |
| Reef Relief | | | | | relief treatments. The control reef |
| Treatment Low | −0.7400769 | 0.5095672 | −1.452364 | 0.1544 | safe sunscreen is having a negative |
| Reef Relief | | | | | effect on the weight growth of the |
| Treatment | −1.1410000 | 0.5289514 | −2.157098 | 0.0372 | Acroporas (t = −2.157 and p = 0.0372.) |
| Sunscreen | | | | | |
| Control | | | | | |
| (Intercept) | 2.1120000 | 0.2211246 | 9.551176 | 0.0000 | There are no significant differences |
| Treatment | 0.5850000 | 0.5068307 | 1.154232 | 0.2554 | between any of the treatments and |
| Control | | | | | the high reef relief doses in the |
| Treatment Low | −0.1550769 | 0.3171333 | −0.488996 | 0.6276 | Acropora's (p > 0.05 for all |
| Reef Relief | | | | | comparisons). |
| Treatment | −0.5560000 | 0.3474253 | −1.600344 | 0.1176 | |
| Sunscreen | | | | | |
| Control | | | | | |
| (Intercept) | 1.9569231 | 0.2273267 | 8.608417 | 0.0000 | There are no significant differences |
| Treatment | 0.7400769 | 0.5095672 | 1.452364 | 0.1544 | between any of the treatments and |
| Control | | | | | the low reef relief doses (p > 0.05 for |
| Treatment High | 0.1550769 | 0.3171333 | 0.488996 | 0.6276 | all comparisons.) |
| Reef Relief | | | | | |
| Treatment | −0.4009231 | 0.3514053 | −1.140914 | 0.2609 | |
| Sunscreen | | | | | |
| Control | | | | | |

TABLE 14
Montipora Weight Results

| | Value | Std. Error | t-Value | p-Value | Notes |
|---|---|---|---|---|---|
| (Intercept) | 1.3360000 | 0.1975538 | 6.762715 | 0.0000 | There is no significant difference |
| Treatment High | 0.3160000 | 0.4398191 | 0.718477 | 0.4765 | between the control and any of the |
| Reef Relief | | | | | treatments (p > 0.05 for all |
| Treatment Low | 0.3433333 | 0.2604549 | 1.318206 | 0.1948 | treatments.) |
| Reef Relief | | | | | |
| Treatment | 0.1630000 | 0.2619182 | 0.622332 | 0.5372 | |
| Sunscreen | | | | | |
| Control | | | | | |
| (Intercept) | 1.3360000 | 0.1975538 | 6.762715 | 0.0000 | There is no significant difference |
| Treatment High | 0.3160000 | 0.4398191 | 0.718477 | 0.4765 | between the high reef relief |
| Reef Relief | | | | | treatment and any of the treatments |
| Treatment Low | 0.3433333 | 0.2604549 | 1.318206 | 0.1948 | (p > 0.05 for all treatments.) |
| Reef Relief | | | | | |
| Treatment | 0.1630000 | 0.2619182 | 0.622332 | 0.5372 | |
| Sunscreen | | | | | |
| Control | | | | | |
| (Intercept) | 1.6793333 | 0.1697329 | 9.893976 | 0.0000 | There is no significant difference |
| Treatment | −0.3433333 | 0.2604549 | −1.318206 | 0.1948 | between the low reef relief |
| Control | | | | | treatment and any of the treatments |
| Treatment High | −0.0273333 | 0.4280450 | −0.063856 | 0.9494 | (p > 0.05 for all treatments.) |
| Reef Relief | | | | | |
| Treatment | −0.1803333 | 0.2416255 | −0.746334 | 0.4597 | |
| Sunscreen | | | | | |
| Control | | | | | |

TABLE 15
Galaxea Weight Results

| | Value | Std. Error | T-Value | P-Value | Notes |
|---|---|---|---|---|---|
| (Intercept) | 2.4750000 | 0.3257874 | 7.596978 | 0.0000 | There is no significant difference |
| Treatment High | 0.0350000 | 0.4550999 | 0.076906 | 0.9389 | between the control and any of the |
| Reef Relief | | | | | treatments (p > 0.05 for all |
| Treatment Low | 0.1403333 | 0.3587565 | 0.391166 | 0.6966 | treatments.) |
| Reef Relief | | | | | |
| Treatment | −0.7070000 | 0.4133443 | −1.710438 | 0.0908 | |
| Sunscreen | | | | | |
| Control | | | | | |
| (Intercept) | 2.5100000 | 0.3177711 | 7.898768 | 0.0000 | There is no significant difference |
| Treatment | −0.0350000 | 0.4550999 | −0.076906 | 0.9389 | between the sunscreen control and |
| Control | | | | | any of the treatments (p > 0.05 for |
| Treatment Low | 0.1053333 | 0.3514929 | 0.299674 | 0.7651 | all treatments.) |
| Reef Relief | | | | | |
| Treatment | −0.7420000 | 0.4070559 | −1.822845 | 0.0718 | |
| Sunscreen | | | | | |
| Control | | | | | |
| (Intercept) | 2.6153333 | 0.1502291 | 17.408971 | 0.0000 | There is a significant difference |
| Treatment | −0.1403333 | 0.3587565 | −0.391166 | 0.6966 | between the low reef relief dose |
| Control | | | | | and sunscreen control (t = −2.868, |
| Treatment High | −0.1053333 | 0.3514929 | −0.299674 | 0.7651 | p = 0.0052.) This indicates a |
| Reef Relief | | | | | significant increase in weight |
| Treatment | −0.8473333 | 0.2954401 | −2.868038 | 0.0052 | growth in the low dose of reef relief |
| Sunscreen | | | | | treatment compared to the control |
| Control | | | | | sunscreen treatment. There is no |
| | | | | | difference between the other |
| | | | | | treatments. |

TABLE 16
Porites Weight Results

| | Value | Std. Error | T-Value | P-Value | Notes |
|---|---|---|---|---|---|
| (Intercept) | 1.8755000 | 0.1841320 | 10.185627 | 0.0000 | There is no significant difference |
| Treatment High | 0.0845000 | 0.2846624 | 0.296843 | 0.7673 | between the control and any of the |
| Reef Relief | | | | | treatments (p > 0.05 for all |
| Treatment Low | −0.3048333 | 0.2087366 | −1.460373 | 0.1478 | treatments.) |
| Reef Relief | | | | | |
| Treatment | −0.1345000 | 0.2705819 | −0.497077 | 0.6204 | |
| Sunscreen | | | | | |
| Control | | | | | |
| (Intercept) | 1.9600000 | 0.2170900 | 9.028515 | 0.0000 | There is no significant difference |
| Treatment | −0.0845000 | 0.2846624 | −0.296843 | 0.7673 | between the low reef relief |
| Control | | | | | treatment and any of the |
| Treatment Low | −0.3893333 | 0.2383158 | −1.633687 | 0.1060 | treatments (p > 0.05 for all |
| Reef Relief | | | | | treatments.) |
| Treatment | −0.2190000 | 0.2940035 | −0.7448899 | 0.4584 | |
| Sunscreen | | | | | |
| Control | | | | | |
| (Intercept) | 1.5706667 | 0.09831769 | 15.975423 | 0.0000 | There is no significant difference |
| Treatment | 0.30483333 | 0.20873660 | 1.460373 | 0.1478 | between the high reef relief |
| Control | | | | | treatment and any of the |
| Treatment High | 0.3893333 | 0.23831580 | 1.633687 | 0.1060 | treatments (p > 0.05 for all |
| Reef Relief | | | | | treatments.) |
| Treatment | 0.1703333 | 0.22130601 | 0.769673 | 0.4436 | |
| Sunscreen | | | | | |
| Control | | | | | |

Various embodiments of the present invention were tested and the testing and results are detailed above in Tables 13 through 16 and discussed below. The sunscreen compositions 100 of the present invention are referred to during research as “Reef Relief” and/or “Coral Care.” The results showed that the effect of the tested embodiments of the sunscreen compositions 100 differed depending on the species, genus, or type of coral tested.

Research Methods. The UV filter in the Reef Relief 30 SPF sunscreen composition 100 was 8.8% titanium dioxide, other than the ingredients added to benefit the corals (including the trace mineral blend 110), the Reef Relief sunscreen base 150 ingredients were identical to the control reef safe sunscreen, including the UV filter and its concentration. Two tanks acted as a control with no additional substances, three were dosed with a low concentration of 30 SPF Reef Relief sunscreen (75 μL⁻¹), two were dosed with a high concentration of 30 SPF Reef Relief sunscreen (6.3 mgL⁻¹) and two were dosed with a high concentration of 30 SPF reef safe control sunscreen (6.3 mgL⁻¹). The high concentration treatment was selected as a result of previous researchers' testing of titanium dioxide as a reef safe UV filter (Corinaldesi, C., Marcellini, F., Nepote, E., Damiani, E. & Danovaro, R. (2018) Impact of inorganic UV filters contained in sunscreen products on tropical stony corals (Acropora spp.). Science of the Total Environment, 637-638, 1279-1285. doi: 10.1016/j.scitotenv.2018.05.108.). The lower concentration was determined as a result of the concentrations of oxybenzone, which was of the lowest detectable concentrations found in areas tested in the field-in this instance the US Virgin Islands (Downs, C. A., Kramarsky-Winter, E., Segal, R., Fauth, J., Knutson, S., Bronstein, O., Ciner, F. R., Jeger, R., Lichtenfeld, Y., Woodley, C. M., Pennington, P., Cadenas, K., Kushmaro, A. & Loya, Y. (2016) Toxicopathological effects of the sunscreen UV filter, oxybenzone (benzophenone-3), on coral planulae and cultured primary cells and its environmental contamination in Hawaii and the U.S. Virgin Islands. Archives of Environmental Contamination and Toxicology, 70 (2), 265-288. doi: 10.1007/s00244-015-0227-7).

Coral Test Species Selection. Four coral species were selected as representative species of tropical reef building corals: Porites lobata, Montipora capricornis, Galaxea fascicularis and Acropora muricata. The coral fragments were prepared onto individual coral plugs at least one month before the experiment began. They were further acclimated in the experimental tanks for four days prior to the first dose. Each tank contained 30 coral fragments, five A. muricata and M. capricornis and ten G. fascicularis and P. lobata (five each of two different morphologies). The corals were fed 15 ml of phytoplankton thrice weekly with two cubes of frozen brine shrimp split evenly between each tank.

Tank Parameters. Nine identical 203 L tanks filled with artificial seawater were used in this experiment, heated to 26.0c+/−0.5c monitored with HOBO data loggers and a HANNA salinity tester, with the salinity kept between 32-35 ppt. Each tank had 10 L water changes occur twice weekly and was dosed with 10 ml of Kh buffer thrice weekly, 10 ml of Ca and Mg buffer and 0.2 ml of I weekly. Water tests were undertaken weekly testing Ca, Mg, Ph, PO4. Kh, NO2, NO3 and NH3 levels within each tank. The tanks were exposed to light in a 12 hr: 12 hr diurnal light: dark cycle at 300 μmol photons m⁻² s⁻¹.

Data Collection. The wet weight of each coral was measured fortnightly to a resolution of 0.01 g, with photographs taken for 2D photometry. The photographs were then used to calculate the linear growth of the coral fragments using ImageJ (Rasband, 2024).

Heat Stress. At week 16, two of each coral species and morphology were taken from each tank and stored in the −80c freezer in individual plastic bags for lipid analysis, with a small fragment taken from each, which was stored in 100% ethanol for analysis of the microbiome using 16S rRNA sequencing. Temperatures were then raised to cause heat stress from week 16 to 18 to monitor whether the addition of the Reef Relief sunscreen has any impact on the resilience of corals when faced with rising temperatures. Each tanks temperature was raised gradually until they reached 30° C. (Santoro et al., 2021). After the tanks reached 30° C. they remained at that temperature for six days. Each coral was monitored for paling and tissue loss throughout the heat stress event; color shifts was monitored using the Coral Watch coral health chart.

Analysis. Analysis was undertaken in R (R Core Team, 2021). Coral weights were analyzed using a linear regression with a GLS (generalized least squares framework) extension. The preliminary linear growth measurements were analyzed using a mix of linear regressions and linear regressions with GLS extensions to account for heterogeneity of variance.

Results on Coral Health. The Acropora fragments in tank 6, one of the low Reef Relief treatment tanks experienced a disease which was first noted in week 10. By the end of week 16 all five of the Acropora fragments in this tank experienced full mortality, this was not repeated in any of the other tanks. Tank 4, a sunscreen control tank, experienced a crash causing paling in all corals in week 13, prior to that five fragments of Acropora were experiencing gradual tissue loss, which started in week 8. Prior to this one other coral was experiencing tissue loss and one had paled tissue. By week 16 the sunscreen control was experiencing the most tissue loss, followed by the high Reef Relief treatment (see Table 17).

TABLE 17
Number of coral fragments (across all species tested)
experiencing paling or tissue loss in week 16,
excluding the tissue loss caused by disease.

| | Paling | Tissue Loss |
|---|---|---|
| Control | 0 | 0 |
| Low Reef Relief | 0 | 1 |
| High Reef Relief | 5 | 2 |
| Sunscreen Control | 31 | 7 |

Results on Weight. The weights were analyzed using a GLS in R (R Core Team, 2021). The weight of the Galaxea fragments significantly increased by approximately 17% (Table 18) in both the low and high Reef Relief treatments in comparison to those located in the control sunscreen treatments (p=0.0052, t=2.868). However, average weight was the same as control fragments suggesting no additional benefit to the corals but no adverse effect.

For Acropora, all treatments showed reduced growth (wet weight) compared to controls. The control sunscreen appeared to have the highest impact on weight showing 15.9% less than non-treated controls (Table 17) and this change was significant (p=0.0372 and t=−2.157).

Monitpora showed an increase in growth across all treatments, with low Reef Relief performing better, however these results were not significant (p=0.1948, t=1.318206). For Porites, both the control sunscreen and low Reef Relief showed a non-significant reduced growth rate (p=0.6204, t==0.49707 and p=0.1478, t=−1.460373 respectively), but the high Reef Relief treatment very marginally increased growth, but again was non-significant (p=0.7673, t=0.0296843).

TABLE 18
Mean percentage increase in the weight of each coral genus within
each treatment from week 2 to week 16. “(*)” represents
a decrease in growth from controls, whilst “(#)” shows an increase.

| | | Control | | |
|---|---|---|---|---|
| | Control | Sunscreen | Low Reef Relief | High Reef Relief |
| Acropora | 38.24% | 22.34% (*) | 25.97% (*) | 30.69% (*) |
| Montipora | 18.98% | 19.24% (#) | 22.44% (#) | 20.32% (#) |
| Galaxea | 43.21% | 27.74% (*) | 44.91% (#) | 43.28% (#) |
| Porites | 28.79% | 24.16% (*) | 24.12%(*) | 28.84% (#) |

Results on Linear Growth. The results for linear growth were analyzed at week 2 and week 16 as the first and last measurements in phase one of the experiment. Horizontal and vertical measurements of each coral fragment were used as a proxy to monitor growth throughout the experiment.

Acropora fragments across all treatments showed reduced linear growth compared to the controls (Table 13). Interestingly, the low Reef Relief treatment showed the lowest growth rates across all treatments with 63% less than controls. That said, it is worth noting that this treatment experienced a disease outbreak in one of the tanks limiting their opportunity for growth.

In contrast, Montipora fragments in low Reef Relief tanks experienced a 14% increase in horizontal linear growth compared to control fragments. Although this was non-significant (Pr=0.059538, t=1.938), the value is marginal suggesting with higher replication a difference may have occurred. Both the sunscreen control and the high Reef Relief showed reduction in horizontal linear growth, a non-significant (Pr=0.437363, t=−0.784) and significant reduction (Pr=0.072593, t=−1.843) respectively.

Horizontal growth of Galaxea was significantly reduced in the control sunscreen tanks compared to the control tanks (Pr=0.0071, t=−2.762972) but had a non-significant increase in both low Reef Relief (p=0.4146, t=0.819948) and high Reef Relief (p=0.4146, t=0.819948) (Table 9). Similarly, horizontal growth of Porites fragments was significantly reduced by the control sunscreen in comparison to the control fragments (Pr=0.0364, t=−2.125378). But there was only a marginal average percentage increase (3%), in growth in the high Reef Relief treatment and this was not significance and there was no observable change between linear growth of the controls and those in low Reef Relief.

TABLE 19
Mean percentage increase in the horizontal linear growth
of each coral genus within each treatment from week
2 to week 16. “(*)” represents a decrease
in growth from controls, whilst “(#)” shows an increase.

| | | Sunscreen | Low Reef | High Reef |
|---|---|---|---|---|
| | Control | Control | Relief | Relief |
| Acropora | 85.96% | 49.32% (*) | 22.71% (*) | 66.03% (*) |
| Montipora | 38.74% | 25.16% (*) | 52.34% (#) | 12.98% (*) |
| Galaxea | 159.14% | 113.10% (*) | 202.16% (#) | 188.04% (#) |
| Porites | 49.89% | 33.07% (*) | 48.54% (*) | 52.91% (#) |

For vertical growth measurements, Acropora showed a significant decrease across all treatments compared to controls (Table 20). Montipora in contrast showed the complete opposite and results indicate an increase in growth across all treatments with low Reef Relief seeing an increase in vertical growth by upwards of 68.4%. Similarly, Galaxea also appeared to show increased vertical growth for both high Reef Relief (Pr=0.00176, t=3.232) and low Reef Relief (Pr=0.03137, t=2.189), by 38.33% and 29.19% respectively compared to the control fragments. The control sunscreen showed a reduced growth for this species, by nearly half.

TABLE 20
Mean percentage increase in the vertical linear growth
of each coral genus within each treatment from week
2 to week 16. “(*)” represents a decrease
in growth from controls, whilst “(#)” shows an increase.

| | | Sunscreen | Low Reef | High Reef |
|---|---|---|---|---|
| | Control | Control | Relief | Relief |
| Acropora | 63.22% | 31.17% (*) | 22.11% (*) | 16.61% (*) |
| Montipora | 56.34% | 68.15% (#) | 124.70% (#) | 65.39% (#) |
| Galaxea | 42.05% | 26.22% (*) | 71.24% (#) | 80.38% (#) |
| Porites | 90.11% | 118.39% (#) | 94.15% (#) | 86.99% (#) |

So, in general, there was very varied differences across the health metrics assessed and the coral species tested. For Acropora, all treatments performed badly, with negative growth rates compared to controls. For Monitpora, all treatments showed a positive increase in vertical linear growth and weight, but only low Reef Relief showed a positive increase in horizontal linear growth. For Galaxea, the ‘reef safe’ sunscreen consistently unperformed against the control fragments with less growth. In contrast, both the Reef Relief sunscreens showed significant increases in growth metrics.

With Porites, only the high Reef Relief treatment consistency showed an increase in growth across all metrics. Vertical linear growth increased across all treatments with regard to vertical linear growth but for both horizontal linear growth and wet weight, the reef safe control sunscreen was negative compared to the growth.

### Additional Research on One Embodiment of a Sunscreen Composition 100 (“CoralCare”).

This embodiment of the CoralCare 30 SPF sunscreen composition 100 was tested to assess the effects of the CoralCare sunscreen composition 100 on coral health and growth in comparison to a commercially available “reef safe” sunscreen and baseline controls. Results demonstrate that coral responses vary by genus, with CoralCare sunscreen composition 100 treatments generally resulting in neutral or positive impacts on growth metrics. Conversely, the control sunscreen negatively affected coral health and growth across all tested genera.

Overview of Findings. The effect of sunscreens on coral health and growth was found to depend, in part, on the coral genus. CoralCare 30 SPF sunscreen composition 100 showed either no significant differences compared to controls or resulted in statistically significant growth increases in specific metrics for certain genera. The commercially available “reef safe” sunscreen, by contrast, caused tissue loss, paling, and reduced growth in multiple metrics across genera.

The findings indicate that CoralCare 30 SPF sunscreen composition 100 is safe for corals and may provide growth benefits in certain genera, such as Galaxea fascicularis. In contrast, the commercially available “reef safe” sunscreen caused significant harm, emphasizing the need for more stringent evaluation of “reef safe” claims.

Sunscreen Products. The CoralCare 30 SPF sunscreen composition 100 contains 8.8% non-nano titanium dioxide as the UV filter, with additional ingredients designed to benefit corals (a trace mineral blend 110). These were identical to the control sunscreen except for the coral-beneficial components. The following treatment conditions were tested:
Baseline control: No substances added (2 tanks).
Low CoralCare sunscreen composition 100 concentration: 75 μL⁻¹ (3 tanks).
High CoralCare sunscreen composition 100 concentration: 6.3 mgL⁻¹ (2 tanks).
Control sunscreen: 6.3 mgL⁻¹ (2 tanks).

The concentrations for testing were based on field-detected levels of oxybenzone (Downs, C. A., Kramarsky-Winter, E., Segal, R., Fauth, J., Knutson, S., Bronstein, O., Ciner, F. R., Jeger, R., Lichtenfeld, Y., Woodley, C. M., Pennington, P., Cadenas, K., Kushmaro, A. & Loya, Y. (2016) Toxicopathological effects of the sunscreen UV filter, oxybenzone (benzophenone-3), on coral planulae and cultured primary cells and its environmental contamination in Hawaii and the U.S. Virgin Islands. Archives of Environmental Contamination and Toxicology, 70 (2), 265-288. doi: 10.1007/s00244-015-0227-7.) and prior studies on titanium dioxide impacts (Corinaldesi, C., Marcellini, F., Nepote, E., Damiani, E. & Danovaro, R. (2018) Impact of inorganic UV filters contained in sunscreen products on tropical stony corals (Acropora spp.). Science of the Total Environment, 637-638, 1279-1285. doi: 10.1016/j.scitotenv.2018.05.108).

Coral Species. Four coral species were selected to represent varying morphologies and stress tolerances:
Porites lobata (two color morphs)
Montipora capricornis
Galaxea fascicularis
Acropora muricata
   Coral fragments were grown on plugs for at least one month before experimentation and acclimated for a week before treatments began. Each tank contained 30 coral fragments distributed across species.

Tank Parameters. Nine identical 203 L tanks with artificial seawater were maintained at 26° C. (±0.5° C.), with salinity between 32-35 ppt. Tanks received biweekly 10 L water changes and were dosed with buffers (KH, Ca, Mg, and I) to maintain stable water chemistry. A 12-hour light: dark cycle was used, and coral feeding occurred three times per week. Regular water testing (e.g., Ca, Mg, PO4) ensured stable tank conditions.

Data Collection. For growth metrics, wet weight was measured fortnightly, and 2D photometry was used to assess horizontal and vertical growth. Heat stress tests were performed in weeks 16-18, tank temperatures were gradually increased to 30° C. to simulate heat stress, and corals were monitored for paling and tissue loss. Lipid analysis and microbiome sequencing were planned for later stages. For statistical analysis, percentage growth was calculated relative to initial measurements. Data were analyzed using general linear regression and generalized least squares (GLS) frameworks in R (R Core Team, 2021).

Results. For the water chemistry, ICP-OES and N-DOC tests in weeks 13 and 16 showed no significant differences in water chemistry across treatments, indicating that the sunscreen products did not alter tank conditions.

### For Coral Health:

Control Sunscreen: Corals in the control sunscreen treatment showed paling (51.7%) and tissue loss (11.7%) by week 16, with Acropora fragments exhibiting gradual tissue loss starting in week 8.
CoralCare Treatments: Low CoralCare concentration showed minimal impacts (1.1% tissue loss, no paling), while high CoralCare concentration resulted in 8.3% paling and 3.3% tissue loss. These effects were significantly lower than the control sunscreen.

TABLE 21
Coral Health Metrics (Week 16)

| Treatment | Paling (%) | Tissue Loss (%) |
|---|---|---|
| Baseline Control | 0.0 | 0.0 |
| Low CoralCare | 0.0 | 1.1 |
| High CoralCare | 8.3 | 3.3 |
| Control Sunscreen | 51.7 | 11.7 |

### Growth Metrics.

### Wet Weight:

Galaxea fascicularis: Both CoralCare treatments showed non-significant increases in wet weight compared to controls, while the control sunscreen caused a significant reduction (t=−2.218, p=0.0292).
Acropora muricata: Only the control sunscreen significantly reduced wet weight (t=−2.472, p=0.0183). CoralCare treatments showed no significant effects.
Montipora capricornis: Weight increased across all treatments, but differences were not statistically significant.
Porites lobata: No significant differences in wet weight were observed across treatments.
   Linear Growth: Horizontal and Vertical Growth were Measured as Proxies for Coral Expansion:
Galaxea: Low CoralCare treatment significantly increased vertical growth (t=2.409, p=0.018) compared to controls. The control sunscreen caused significant reductions in vertical growth (t=−2.249, p=0.027).
Acropora: No significant differences in horizontal or vertical growth were observed across treatments.
Montipora: Control sunscreen caused significant reductions in horizontal growth (t=−2.522, p=0.016). High CoralCare treatment showed a marginally non-significant reduction (t=−1.893, p=0.066).
Porites: Control sunscreen significantly reduced horizontal growth (t=−2.641, p=0.010), while CoralCare treatments showed no significant effects.

Photographic Evidence. Representative images of Galaxea fragments showed marked improvements in horizontal and vertical growth under low CoralCare treatment compared to both controls and the control sunscreen. Horizontal growth increased by over 21%, vertical growth by 5%, and wet weight by 8% compared to baseline controls. Compared to the control sunscreen, these values doubled for horizontal growth (+50%) and increased by 36% for vertical growth.

Discussion. The tested embodiments of CoralCare 30 SPF sunscreen composition 100 exhibited neutral to positive impacts on coral health and growth compared to baseline controls, with notable benefits observed in Galaxea fascicularis. Importantly, the CoralCare sunscreen composition 100 performed significantly better than the commercially available “reef safe” sunscreen, which negatively affected coral growth and health across all genera tested. Key takeaways include:
1. CoralCare sunscreen composition 100 is Non-Harmful: Across multiple metrics, CoralCare treatments showed no adverse effects on coral health or growth in comparison to baseline controls.
2. Genus-Specific Benefits: Positive growth effects were observed in Galaxea, particularly at low CoralCare sunscreen composition 100 concentrations.
3. Control Sunscreen Concerns: The “reef safe” control sunscreen caused significant reductions in coral health and growth metrics, highlighting the need for rigorous testing of such products.

The findings indicate that CoralCare 30 SPF sunscreen composition 100 is safe for corals and provides growth benefits in certain genera, such as Galaxea fascicularis. In contrast, the commercially available “reef safe” sunscreen caused significant harm, emphasizing the need for more stringent evaluation of “reef safe” claims.

Various Use Environments. The various embodiments of a coral protective sunscreen composition 100 of the present invention have several additional applications beyond coral tourism activities, such as scuba and snorkeling in and around coral reefs. One application is in the realm of marine conservation. The compositions of the present invention can be applied in broader marine ecosystems to protect and enhance the health of other sensitive organisms, such as seagrass beds and mangroves, which are also affected by chemical pollutants. The bioactive ingredients can provide benefits to marine life by reducing oxidative stress and supporting the health of the surrounding ecosystem.

Another application is in aquaculture. Coral-protective sunscreen compositions 100 of the present invention can be used in aquaculture, particularly for facilities cultivating corals for reef restoration projects. By preventing the introduction of harmful chemicals into the water, such sunscreens could help maintain a healthy growth environment for cultivated coral, ensuring better success rates in restoration efforts.

Coral-protective sunscreen compositions 100 of the present invention can be used in wildlife protection. The sunscreen compositions 100 can be useful for the protection of marine species such as sea turtles, fish, and other organisms sensitive to UV radiation or pollutants. The compositions' 100 non-toxic, bioactive nature also can support skin health and overall resilience in animals that are frequently exposed to human activities in marine tourism zones.

Water sports and recreation are another area of use for coral-protective sunscreen compositions of the present invention. For water sports enthusiasts, surfers, and divers, the sunscreen compositions can provide a protective solution for both their skin and the marine environments they frequent. The eco-friendly ingredients ensure minimal ecological impact, appealing to eco-conscious consumers who engage in snorkeling, diving, and other recreational water activities both near reefs and in freshwater environments.

It is to be understood that the invention may assume alternative variations and step sequences, unless specified to the contrary. It also is to be understood that the specific devices and processes illustrated in the attached drawings and described in this specification are simply exemplary embodiments of the invention. Hence, specific dimensions and other physical characteristics related to the embodiments disclosed are not to be limiting.

What is claimed is:

1. A sunscreen composition comprising:
a sunscreen base; and
a trace mineral blend that has a positive effect on at least one species of coral.

2. The sunscreen composition of claim 1, wherein the sunscreen base comprises a UV-blocking composition that is selected from the group consisting of non-nano zinc oxide and titanium dioxide.

3. The sunscreen composition of claim 1, wherein the trace mineral blend comprises chloride, magnesium, sulfate, sodium, and potassium.

4. The sunscreen composition of claim 1, wherein the trace mineral blend comprises:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

5. The sunscreen composition of claim 1, wherein the trace mineral blend comprises:
chloride in an amount from 340-550 mg/g of sunscreen composition,
magnesium in an amount from 140-210 mg/g of sunscreen composition,
sulfate in an amount from 15-60 mg/g of sunscreen composition,
sodium in an amount from 2.5-9 mg/g of sunscreen composition, and
potassium in an amount less than 4 mg/g of sunscreen composition.

6. The sunscreen composition of claim 1, wherein the at least one species of coral is selected from the group consisting of Porites lobata, Montipora capricornis, Galaxea fascicularis and Acropora muricata.

7. A sunscreen composition comprising:
an oil phase comprising
titanium dioxide in an amount from about 2.5% to about 25% or zinc oxide in an amount from about 10% to about 25%,
polyhydroxystearic acid, and
a mixture comprising one or more esters, and
an aqueous phase comprising
water,
a salt,
a trace mineral blend in an amount from 0.1% to 5%,
glycerol,
an aloe compound, and
green tea extract.

8. The sunscreen composition of claim 7, wherein the trace mineral blend comprises:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

9. A sunscreen composition comprising:
an oil phase comprising titanium dioxide in an amount from 2.5% to about 25% or zinc oxide in an amount from 10% to about 25%,
polyhydroxystearic acid,
a mixture comprising one or more esters, and
a trace mineral blend in an amount from 0.1% to 5%.

10. The sunscreen composition of claim 9, wherein the trace mineral blend comprises:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

11. A skin care composition comprising:
a skin care base; and
a trace mineral blend that has a positive impact on at least one species of coral.

12. The skin care composition of claim 11, wherein skin care base is selected from the group consisting of a moisturizer, a toner, an essence, and a treatment.

13. The skin care composition of claim 11, wherein the trace mineral blend comprises chloride, magnesium, sulfate, sodium, and potassium.

14. The skin care composition of claim 11, wherein the trace mineral blend comprises:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

15. A method of making a skin care composition by combining a skin care base and a trace mineral blend, the trace mineral blend comprising:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

16. A method of making a sunscreen composition by combining a sunscreen base oil phase and a sunscreen base aqueous phase, wherein the aqueous phase comprises a trace mineral blend, the trace mineral blend comprising:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

17. A sunscreen composition comprising:
a sunscreen base; and
a trace mineral blend comprising:
chloride in an amount from 200 to 600 ppm,
magnesium in an amount from 100 to 300 ppm,
sulfate in an amount from 2 to 100 ppm,
sodium in an amount from 2 to 100 ppm, and
potassium in an amount from 0 to 100 ppm.

18. The sunscreen composition of claim 17, wherein the sunscreen base comprises a UV-blocking composition that is selected from the group consisting of non-nano zinc oxide and titanium dioxide.

19. The sunscreen composition of claim 17, wherein the trace mineral blend is an amount from 0.1% to 5% weight of the sunscreen composition.

20. The skincare composition of claim 11, wherein the trace miner blend is at least 0.1% weight of the final composition.
